(19) United States
(12) Patent Application Publication
(10) Pub. No.: US 2003/0228295 A1
(43) Pub. Date: Dec. 11, 2003

(54) Use of human neural stem cells secreting GDNF for treatment of parkinson's and other neurodegenerative diseases

(76) Inventor: Clive N. Svendsen, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/423,710

(22) Filed: Apr. 25, 2003

Related U.S. Application Data
(60) Provisional application No. 60/375,587, filed on Apr. 25, 2002.

(51) Int. Cl. A61K 48/00; C12N 5/08

(52) U.S. Cl. 424/93.21; 435/368

(57) ABSTRACT

A method of treating brain disorders involving loss of cells that respond to GDNF is disclosed. In one embodiment, the invention comprises the steps of (a) transducing human neural stem cells with glial-derived neurotrophic factor (GDNF), wherein the GDNF gene is under control of an inducible promoter system, and (b) transplanting the transduced cells into the brain of a patient.

### CROSS-REFERENCE TO RELATED APPLICATION

This application claims priority to U.S. provisional application 60/375,587, filed Apr. 25, 2002, incorporated by reference herein.

### STATEMENT REGARDING FEDERALLY SPONSORED RESEARCH OR DEVELOPMENT

### BACKGROUND OF THE INVENTION

Neurotrophic Factors and Neurological Illness

The degeneration of specific groups of cells in the human brain underlies many devastating diseases such as Parkinson's Disease (PD), Alzheimer's Disease, Huntington's Disease (HD), amyotrophic lateral sclerosis (ALS) and many others. It is also a prime concern for the military due to the prevalence of neurotoxic chemical weapons and war related head injury. PD affects one of every 100 people over 60 or approximately 1.5 million Americans, and costs the US an estimated 25 billion dollars a year. Treatment consists mainly of administering a dopamine precursor L-DOPA. This is very effective in the early stages of the disorder, but later leads to severe side effects and eventually no longer works. Newer agents are being produced to enhance dopamine efficiency, and alternative neurosurgical approaches are also being developed. Here, specific brain regions are either lesioned or stimulated which often results in dramatic acute clinical benefit (The Deep-Brain Stimulation for Parkinson's Disease Study Group, 2001). However, there can also be changes in executive function (Jahanshahi, et al., Brain 123(Pt. 6):1142-1154, 2000) and the long-term prognosis for these patients in the face of ongoing neuronal degeneration is not yet known.

Transplantation of dopamine neurons derived from fetal tissues has also shown great promise in PD. Here, new dopamine neurons integrate into the putamen of patients and provide a new source of dopamine—which in some cases leads to clinical improvement (Dunnett and Bjorklund, Nature 399:A32-A39, 1999). Transplantation actually replaces the neurons lost during the course of the disease, but as they are placed ectopically in the putamen they may not be optimal for clinical recovery. The latest study on a large group of patients which included sham operations has shown that although younger patients respond to the transplants, the effects were less dramatic in older patients and, in some cases, there were side-effects including dyskinesias, even in the absence of L-DOPA (Freed, et al., N. Engl. J. Med. 344:710-719, 2001). However, there is some discussion as to why these side-effects were seen in this study, which used a very different protocol to the many that preceded it (Isacson, et al., Nat. Neurosci. 4:553, 2001). While drugs, neurosurgical methods and transplantation represent real opportunities to improve the quality of life for PD patients, there are other alternatives. The most attractive prospect would be to prevent the loss of dopamine neurons, or to encourage existing cells to put out new processes. In this way the disease is being treated, rather than the symptoms. It is very possible that neuotrophic factors may provide a way of achieving this in the near future.

The archetypical neurotrophic factor is nerve growth factor (NGF), which was shown to regulate the survival and differentiation of developing sympathetic and dorsal root ganglion neurons (Levi-Montalcini and Angeletti, Dev. Biol. 7:653-659, 1963). Following its discovery in 1963, there have been a plethora of new neurotrophic factors that have similar, but nonetheless specific effects. Two structurally and functionally related families have emerged. These are (i) the NGF—super family that includes NGF, BDNF, NT-3, NT-4/5 and NT-6 and (ii) the glial cell-line derived neurotrophic family (GDNF) which includes GDNF, persephin and neurturin. The GDNF family has established neuroprotective effects on dopamine neurons, and enhances neurite outgrowth; both in vitro (Lin, et al., Science 260:1130-1132, 1993) and in vivo following damage (Beck, et al., Nature 373:339-341, 1995; Tomac, et al., Nature 373:335-339, 1995; Bjorklund, et al., Neurobiol. Dis. 4:186-200, 1997). We have previously shown that GDNF can also enhance fiber outgrowth from embryonic dopamine neurons transplanted into a rat model of PD (Sinclair, et al., Neuroreport 7:2547-2552, 1996). Via modulation of the intact dopaminergic system, GDNF may also have a role in adaptations to drugs of abuse (Messer, et al., Neuron 26:247-257, 2000), and as its receptors are found throughout the brain it is also likely to affect a number of other neurotransmitter systems (Golden, et al., J. Comp. Neurol. 398:139-150, 1998). This may be why GDNF can also protect other neurons from cell death in a variety of different models.

The relevance of GDNF to PD was further established through studies involving a unique toxin 1-Methyl-4-phenyl-1,2,3,6-tetrahydropyridine (MPTP), which causes Parkinson's-like symptoms in humans through the selective elimination of dopamine neurons (Langston, Acta Neurol. Scand Suppl. 100:49-54, 1984). GDNF has been shown to protect against MPTP toxicity in both mouse and monkey models when infused directly into the brain parenchyma (Tomac, et al., Nature 373(6512):335-339, 1995; Gash, et al., Nature 380:252-255, 1996). These encouraging studies led to a recent clinical trial where GDNF was infused in bolus injections directly into the cerebral ventricles of PD patients. However, the results were very disappointing with no reduction in rating scores for PD, some side effects and little evidence of restoration of dopamine fibers in the striatum at post mortem (Kordower, et al., Ann. Neurol. 46:419-424, 1999). The reason for this lack of effect was very likely due to lack of GDNF penetration into the brain.

A number of years ago, NGF (which is a similar size) was shown to penetrate very poorly into the brain parenchyma following intra-ventricular injection (Lapchak, et al., Neuroscience 54:445-460, 1993). Thus the likelihood of GDNF actually reaching a deep structure such as the putamen was very low.

Viruses as Delivery Agents for Therapeutic Genes

The use of engineered, replication-deficient viruses to transduce brain cells has been established. These have been modified to drive GDNF under a variety of promoters (reviewed in Bjorklund, et al., Brain Res. 886:82-98, 2000). In nearly every study, GDNF was found to both protect dopamine neurons and enhance fiber outgrowth. Our collaborators have developed a self-inactivating version of lenti-GDNF that is safe, non-toxic and expresses the transgene for extended periods of time (Deglon, et al., Hum. Gene Ther. 11:179-190, 2000). Furthermore, this same virus has been shown to reverse age-induced reductions in dopaminergic expression, and prevent MPTP toxicity following direct injection to the striatum of rhesus monkeys (Kordower, et al., Science 290:767-773, 2000). As such, it represents great potential as a delivery system for GDNF to the brain of PD patients.

Ex Vivo Gene Therapy and Inducible Viral Vectors

An alternative approach is ex vivo gene therapy. Fibroblasts, astrocytes or other cell lines are first transduced with the gene of interest, and then transplanted into the brain (for review see Gage, Nature 392(supplement):18-24, 1998). Cells which may be tumerigenic or likely to induce an immune response can be placed in capsules that prevent their escape and detection while allowing protein diffusion through a permeable membrane (Tseng and Aebischer, Prog. Brain Res. 127:189-202, 2000). GDNF released from such encapsulated cells can restore function and increase dopamine metabolism in aged rats (Emerich, et al., Brain Res. 736:99-110, 1996). However, for human clinical trials there would be an advantage to moving away from permanent indwelling devices if possible. Furthermore, capsule delivery of GDNF still represents a point source of protein delivery, rather than a diffuse delivery across a wider area. Ideally, the cells would be transplanted into the brain, migrate within the desired target region and release GDNF in the milieu of the degenerating nerve fibers or cells. This technique would overcome problems highlighted above in that (i) no host neurons would be genetically modified, (ii) the cells would not harbor live virus and (iii) exact release rates of GDNF could be established in vitro prior to transplantation. However, it is essential in these studies to be able to regulate GDNF release.

The control of GDNF release following grafting remains a serious issue. In any clinical delivery trial there must be a way to turn off the gene of interest, allowing gene regulation if unwanted side effects occurred, or the maximal effect of GDNF was established. Furthermore, it would allow regulation of GDNF release over time and adjustment of exact amounts delivered to the brain in a similar fashion to normal drug delivery. Inducible gene expression systems have now been developed which allow controlled regulation of genes (Blau and Rossi, Proc. Natl. Acad. Sci. USA 96:797-799, 1999). Viral constructs incorporating the tetracycline inducible element have recently been tested. The gene of interest is switched on or off depending on the design of the construct following administration of doxycycline (an analogue of tetracycline) to the culture media in vitro or the drinking water in vivo. These systems have been shown to regulate neurotrophin and GFP production in fibroblasts in vitro (Blesch, et al., J. Neurosci. Res. 59:402-409, 2000), the release of GABA from cell lines in vitro and in vivo after transplantation into rodent models of PD (Berhstock, et al., J. Neurosci. Res. 60(3):302-310, 2000; Behrstock, et al., Sco. For Neurosci., 2001) and GFP production in human HEK 293 cell lines (Kafri, et al., Mol. Ther. 1:516-521, 2000). Furthermore, controlled release of NGF in vivo has been shown to modulate the survival and outgrowth of injured cholinergic neurons (Blesch, et al., Gene Ther. 8:954-960, 2001).

Neural Stem Cells for Ex Vivo Gene Therapy

During the development of the central nervous system (CNS), there is extensive proliferation of neuroepithelial cells lining the ventricular walls which give rise to the neurons, astrocytes and oligodendrocytes of the mature brain (Jacobson, “The germinal cell, histiogenesis, and lineages of nerve cells,” In: Developmental Neurobiology (Jacobson, ed.), New York and London: Plenum Press, 1991). These cells can be isolated in culture and grown as either monolayers or free-floating aggregates termed “neurospheres” (Gage, Science 287:1433-1439, 2000; McKay, Science 276:66-71, 1997; Reynolds and Weiss, Dev. Biol. 175:1-13, 1996; Scheffler, et al., Trends Neurosci. 22:348-357, 1999). Neurospheres probably consist of low numbers of “true” stem cells and many more restricted progenitors (Svendsen, et al., Trends Neurosci. 22:357-364, 1999; Svendsen and Caldwell, Prog. Brain Res. 127:13-34, 2000). Because they can be grown in culture for long periods, and retain the ability to survive transplantation, neurospheres represent the ideal source of tissue for cell therapy (Svendsen and Smith, Trends Neurosci. 22:357-364, 1999).

Neurospheres generated from a transgenic mouse over-expressing NGF secrete biologically active NGF following transplantation (Carpenter, et al., Exp. Neurol. 148:187-204, 1997). Human neural precursor cells have also been infected with adenoviral vectors driving a tetracycline inducible tyrosine hydroxylase (TH) gene. Although the authors reported regulation of the gene both in vitro and in vivo following grafting, the behavioral effects were transient (Corti, et al., Nat. Biotechnol. 17:2349-354, 1999). This may be because non-specific expression of TH is not functionally relevant in many cases.

In other studies, similar human neural precursors have been infected with tetracycline inducible systems driving immortalizing agents (Sah, et al., Nature Biotech. 15:574-580, 1997). Very recently, an immortal cell line was modified to release GDNF and was shown to reverse some of the changes associated with a mouse model of PD (Akerud, et al., J. Neurosci. 21:8108-8118, 2001).

Transplantation of Neurospheres

In parallel to these in vitro studies, we have published a triad of papers concerning the fate of transplanted neural cells. The first paper showed that transplantation of cells from both rat and human cells derived from the developing brain did not generate large grafts similar to those seen using primary fetal tissue, although good markers to follow cells were not available at this stage (Svendsen, et al Exp. Neurol. 137:376-388, 1996). Subsequent studies from other groups showed similar small diffuse grafts with many migrating cells following the transplantation of human cells (Vescovi, et al., Exp. Neurol. 156:71-83, 1999; Fricker, et al., J. Neurosci. 19:5990-6005, 1999). Our next transplant paper, using specific human markers, showed that the majority of cells migrated from the site of injection and matured into astrocytes following transplantation into a rodent model of PD. However, a small number of cells differentiated into dopamine neurons and reversed a rotational deficit in a few animals (Svendsen, et al., Exp. Neurol. 148:135-146, 1997). In our most recent paper, we have established the optimal cell density when grafting human neurosphere cultures, and used a human specific neurofilament marker to demonstrate extensive axonal outgrowth from the transplant (Ostenfeld, et al., Exp. Neurol. 164:215-226, 2000).

### BRIEF SUMMARY OF THE INVENTION

In one embodiment, the present invention is a method of treating brain disorders involving loss of cells that respond to GDNF comprising the steps of (a) transducing human neural stem cells with glial-derived neurotrophic factor (GDNF), wherein the GDNF gene is under control of an inducible promoter system, and (b) transplanting the transduced cells into the brain of a patient.

In a preferred version of the present invention, the patient is selected from a group consisting of Parkinson's Disease patient, ALS patient, stroke patient and Huntington's Disease patient. In another preferred version of the present invention, the inducible promoter is part of the mouse phosphoglycerate kinase 1/tTA1 system.

Other objects, advantages and features of the present invention are described below.

### BRIEF DESCRIPTION OF THE SEVERAL VIEWS OF THE DRAWINGS

FIG. 1 is a diagram of a preferred preparation of neurospheres.

FIG. 2 is a diagram of lentiviral constructs providing regulatable expression of GDNF or reporter gene.

FIGS. 3A-D are photographs of human neural cells infected by a preferred viral construct of the present invention. FIGS. 3A-C represent, respectively, progenitor cells, neurons and astrocytes infected with the ^{ind}lenti-GFP construct. FIG. 3D illustrates cells infected with the ^{ind}lenti-GDNF construct.

FIG. 4 is a set of photographs illustrating GFP regulation. FIGS. 4A-C demonstrate the continued normal growth of the neurosphere over time. FIG. 4D represents infection of neurospheres with the ^{ind}lenti-GFP construct resulting in a high percentage of GFP expressing cells. FIG. 4E demonstrates that when GFP expressing neurospheres were grown in the presence of doxycycline for 48 hours, GFP was almost entirely shut off. Doxycycline was then removed for 48 hours and the robust expression of GFP resumes, as illustrated by FIG. 4F.

FIGS. 5A and B is a set of bar graphs illustrating that GDNF from human neurospheres infected with ^{ind}lenti-GDNF is regulated in a time-dependent fashion. FIG. 5A represents GDNF levels. FIG. 5B represents GDNF levels in the presence of doxycycline.

FIGS. 6A, B and C demonstrate the number of TH-positive cells, length of TH-positive neurites and area of TH-positive cell body, respectively, in basal media, wild-type supernatant and ^{ind}lenti-GDNF supernatant. FIGS. 6D and E demonstrate the functional effects of ^{ind}lenti-GDNF-infected neurospheres (FIG. 6E) compared to wild-type neurospheres (FIG. 6D).

### DETAILED DESCRIPTION OF THE INVENTION

Glial derived neurotrophic factor (GDNF) is a candidate therapeutic for Parkinson's Disease (PD). It can prevent the loss of dopamine neurons in various models of PD and has shown encouraging clinical results and a good safety profile in a recent small clinical trial. GDNF is too large to cross the blood brain barrier and therefore novel methods of delivery need to be developed. Furthermore, its delivery needs to be targeted to specific regions of the brain, as it might have unwanted effects on some neural systems.

In one embodiment, the present invention is a method of treating neurological diseases involving loss of cells that respond to GDNF, such as Parkinson's Disease, comprising the steps of (a) transducing human neural step cells with glial-derived neurotrophic factor (GDNF), wherein the GDNF gene is under control of an inducible promoter system, and (b) transplanting the transduced cells into the brain of a patient. GDNF is expressed and the GDNF-responsive neuron system is up-regulated.

This present invention is based on the use of genetically modified human neural stem cells (hNSC) grown using a novel passaging method as vehicles for targeted delivery of GDNF to specific regions of the brain. The release of GDNF is under control of an inducible promoter system. The cells can be grown in large numbers, and the GDNF released has a biological effect on dopamine neurons which are known to die in Parkinson's disease.

Applicants discuss the various aspects of the present invention below.

Neural stem cells: We have refined techniques for the growth, differentiation and transplantation of human neural stem cells (hNSC). (Svendsen, et al., J. Neurosci. Methods 85(2):141-152, 1998; Svendsen, et al., Brain Pathology 9(3):499-513, 1999 both incorporated by reference.) Preferably, the cells are not derived from human ES cells. Instead, they come from germinal zones of post mortem fetal brain tissue. We collected tissue from the NIH-funded Birth Defects Laboratory, Washington, USA. The advantage of these cells is that they are restricted to producing neural tissue only and do not produce teratomas or other tissue types which is currently a major concern with more primitive ES cell derivatives. It is possible to get cells from a number of different locations such as hospitals or health care centers that can provide miscarriage tissue.

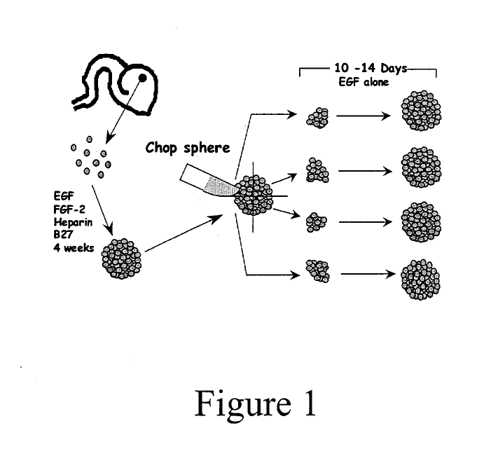

Recently we have shown that hNSCs can be maintained as aggregates termed “neurospheres” for extended periods of time in the presence of EGF/LIF and reach a stable phase of growth between 30-100 population doublings using a novel method of passaging. This method involves “chopping” the spheres into smaller segments rather than using enzymes, thereby maintaining cell/cell contact and the stem cell “niche”. This in turn allows long term growth without addition of complex supplements to the media and the production of cells with a consistent phenotype that can be frozen and banked. In our hands these cells do not form tumors following transplantation. The cells migrate short or long distances, survive for long periods of time and produce both astrocytes and neurons. FIG. 1, discussed in more detail below, describes a preferable method for producing neurospheres.

Using different pre-differentiation methods we have been able to direct the phenotype of cells derived from hNSC into either neurons or glia and control their migration. We have now established a bank of these cells that have undergone (i) extensive tests for adventitious agents, (ii) full karyotypic analysis and (iii) full micro array gene analysis. These cells are publicly available through Clonetics. One with skill in the art could grow the cells using previously published papers (e.g., Svendsen, et al., supra, 1998).

Parkinson's disease (PD) and stem cells. Traditional stem cell approaches to PD have focused on the generation of dopamine neurons from stem cells. This is based on the fact that over 300 PD patients have now been transplanted with primary dopamine neurons from fetal tissue. However, it is now evident that ectopic transplantation of dopamine neurons from primary human fetal tissue into the striatum may not be sufficient to relieve the symptoms of PD in humans. In fact, these cells may induce “off” dyskinesias which are difficult to control. Although speculative, it is possible that these are due to non-controlled release of dopamine in the striatum via small “hot spots” of dopamine neurons within the graft that are not controlled by any efferent connections. Although it is clearly important to continue refining dopamine neuron transplants, further work in primates is now required before moving back to the clinic. Human ES cells are likely to be the best source of dopamine neurons for these studies, as neural stem cells from fetal brain tissue do not readily make dopamine neurons.

Glial derived neurotrophic factor (GDNF): GDNF was discovered through its trophic effects on dopamine neurons in the culture dish. Since then it has been used in a large number of studies to prevent the degeneration of dopamine neurons and support transplanted dopamine neurons in models of PD. We have just completed a clinical trial in the United Kingdom which involved infusion of high concentrations of GDNF into the putamen of 5 PD patients directly using Medtronics pumps. Gill, et al., 2003, infra. Although an open trial, there have been significant clinical improvements in these patients, reductions in dyskinesias and significant increases in dopamine storage in the brain. At the 2 year time point, all patients have tolerated this high dose well and continue to improve. The problem with this approach is that installing the pumps is complicated, the GDNF has to be re-filled every month, the region of the brain infused is small, and there is a chance of infection over long periods of delivery. Furthermore, the cost of GDNF may be prohibitive in the long term.

GDNF delivery using viral vectors. One alternative to pump delivery of GDNF involves viral modification of host cells (in vivo) to release this growth factor. While direct gene therapy is an attractive idea, there remain serious practical and safety issues that include:

Inability to exactly control gene dosing following in vivo delivery

Inability to control exact gene insertion site that from recent reports may be of great importance.

Forcing degenerating cells to express genes of interest may lead to problems as the disease progresses.

Safety issues regarding direct injection of live HIV or other viral types

The approach of the present invention is to modify cells in the culture dish (ex vivo) to produce the growth factor of interest and then transplant these cells into the brain. With this approach:

Cells can be selected for gene dosing (protein release) prior to transplantation.

The exact insertion site can be documented from cloned cells and checked for interference with oncogenes.

The healthy ex vivo cells will provide the protein delivery, not degenerating host cells.

As viral infection takes place in vitro followed by extensive expansion in the absence of virus there is no danger of live virus transfer to the host.

One problem with ex vivo gene therapy has been the type of ex vivo cells used. While autologous fibroblasts would appear to be ideal there are problems. The cells have to be individually manufactured from each patient requiring extensive and expensive culture work to test for gene expression, adventitious agents and purity. When transplanted, fibroblasts will form a “scar” like structure and not migrate to fill a structure, or integrate into the host CNS well. Astrocytes might be another source of cells. However, following expansion human astrocytes are known to lose much of their plasticity following grafting and also form a glial scar structure without good integration and migration patterns.

We suggest here that human neural stem cells may be the ideal vehicle for ex vivo gene therapy for the following reasons:

Neural stem cells can be grown in large numbers.

Neural stem cells generate immature astrocytes which can migrate and integrate.

As they divide in culture, they can be easily infected with viruses.

There is a large literature on successful transplantation of these cells to the brain.

Combining human neural stem cells with gene therapy approaches presents a real opportunity to translate basic science into the clinic. Here, the cells will be used as mini-pumps for various therapeutic proteins.

Preferably, the method of the present invention is accomplished by creating a vector wherein the GDNF gene is under inducible promoter control in a viral system. Preferably, one would use the viral construct we disclose below. Our inducible construct is based on a lentiviral system published in detail previously (Deglon, et al., Hum. Gene Ther. 11:179-190, 2000, incorporated by reference). When we refer to the “mouse phosphoglycerate kinase 1/tTA1 system” we are referring to the promoter system described in Deglon, et al. and below. Of course, one may modify the system by introducing an alternative inducible promoter such as those described below.

One would then transduce human neural stem cells with the GDNF vector, preferably as described below in Materials and Methods.

Translation to the clinic. Our knowledge base for hNSCs has now reached a point where we can describe a clinical application. A major feature of the current invention is the combination of gene therapy with stem cell therapy to produce cells that can act both as replacement vehicles and “mini pumps” for therapeutic proteins. This represents a new and very powerful approach to the treatment of neurological disorders. The cells would be generated as described above and transplanted into the putamen of PD patients.

Patient with PD typically lose dopamine neurons in a topographical fashion from the mesencephalon over time. The first cells to die are those that innervate the caudal regions of the putamen as evidenced by PET scanning methods (Gill, et al., infra, 2003). We envisage targeting the caudal half of the putamen in patients using approximately 4 sites evenly dispersed through this region. Sterotaxic methods, PET techniques and other methods for human trials have been described in detail in Gill, et al., Nature Med., 2003, Mar. 31, 2003, 12669033.

There are two ways in which the inducible promoter system could be used in this invention. The first is in the “on” format, where administration of doxycyline to the patient (which penetrates the blood brain barrier) would activate the GDNF gene construct to induce GDNF release from the transplanted stem cells. If GDNF was found to be safe in the first cohort of patients, we would design a second similar “off” system in which administration of doxycycline to patients would shut off GDNF expression. We predict from our first clinical trial that long term expression of GDNF will not be toxic and so favor the “off” system, which will not require the patient take continual doxycyline to maintain GDNF expression.

Here the cells would integrate into the host brain and release GDNF. The GDNF would be taken up by surrounding dopamine fibers and transported back to the cell bodies in the brain stem. Based on animal studies this should do three things: (i) prevent the ongoing death of dopamine neurons, (ii) induce local fiber outgrowth and (iii) upregulate dopamine production. Together this represents a real “cure” for Parkinson's disease, and in addition would prevent further degeneration of dopamine neurons.

We envision that the stem cell transplants will provide (1) trophic and structural support for sick and dying neurons in PD and other diseases involving loss of cells that respond to GDNF through constitutive release of growth factors and uptake of possible toxins such as glutamate and (2) release of GDNF through the inducible construct. The cellular outcome in PD can be broken into three parts: (1) Up-regulation of the dopaminergic system through direct regulation of dopamine release from terminals; (2) local sprouting of dopamine fibers in the location from the remaining dopamine neurons in the substantia nigra; (3) long term protection of remaining dopamine neurons through retrograde transport of GDNF to cell bodies in the substantial nigra. We expect parallel response in other disease systems (ALS, stroke, HD).

Other neurological diseases: Although PD is an obvious immediate target for stem cell gene therapy, this method of the present invention is applicable to a number other brain disorders involving loss of cells that respond to GDNF. Of these amyotrophic lateral sclerosis (ALS), Huntington's disease (HD) and stroke are the most likely targets. It is not difficult to replace the GDNF transcript with other growth factor transcripts such as ciliary neurotrophic factor (CNTF) and brain-derived neurotrophic factor (BDNF) which may have different but complementary effects to GDNF. Dual infection of hNSC would thus provide a cocktail of growth factors to treat more complex disorders. Neurons which die in Huntington's Disease (HD), stroke and amyotrophic lateral sclerosis (ALS) have all been shown to respond to GDNF treatment. However, it is also possible that combining GDNF with other growth factors may be better for certain diseases. CNTF for example has been shown to have powerful effects on motor neurons that die in amyotrophic lateral sclerosis (ALS)—and so combining with GDNF may be very beneficial.

### EXAMPLES

Materials and Methods

Viral constructs. One common inducible system involves a constitutive promoter driving the tetracycline transactivator (tTA). In the absence of doxycycline (DOX), the tTA binds to an inducible promoter (tetO) located upstream of a minimal promoter which in turn drives the target gene (Gossen and Bujard, Proc. Natl. Acad. Sci. USA 89:5547-5551, 1992). DOX binds tTA and thus prevents transcription of the gene. Another system is the reverse tet-regulated system, which allows gene activation in the presence of doxycycline. Here a mutated form of tTA called rtTA is expressed. rtTA only activates tetO and gene expression when doxycycline is present (Gossen, et al., Science 268:1766-1769, 1995). A more recent method for inducible gene expression utilizes a tTA-KRAB repression system (Freundlieb, et al., J. Gene Med. 1:4-12, 1999). In this tet-on system, the rtTA is bound to the active repressor KRAB. Aside from tetracycline inducible systems, other inducible systems involving glucocorticoids can be used for gene regulation. For instance, the insect steroid horomone ecdysone and the ecdysone receptor fused to an activation domain has provided an inducible gene expression system in mammalian cells and transgenic mice (No, et al., Proc. Natl. Acad. Sci. USA 93:3346-3351, 1996). Also, mifepristone (RU486) and a mutant of the human progesterone receptor fused to an activation domain have been used for inducible gene expression (Wang, et al., Proc. Natl. Acad. Sci. USA 91:81806-81884, 1994).

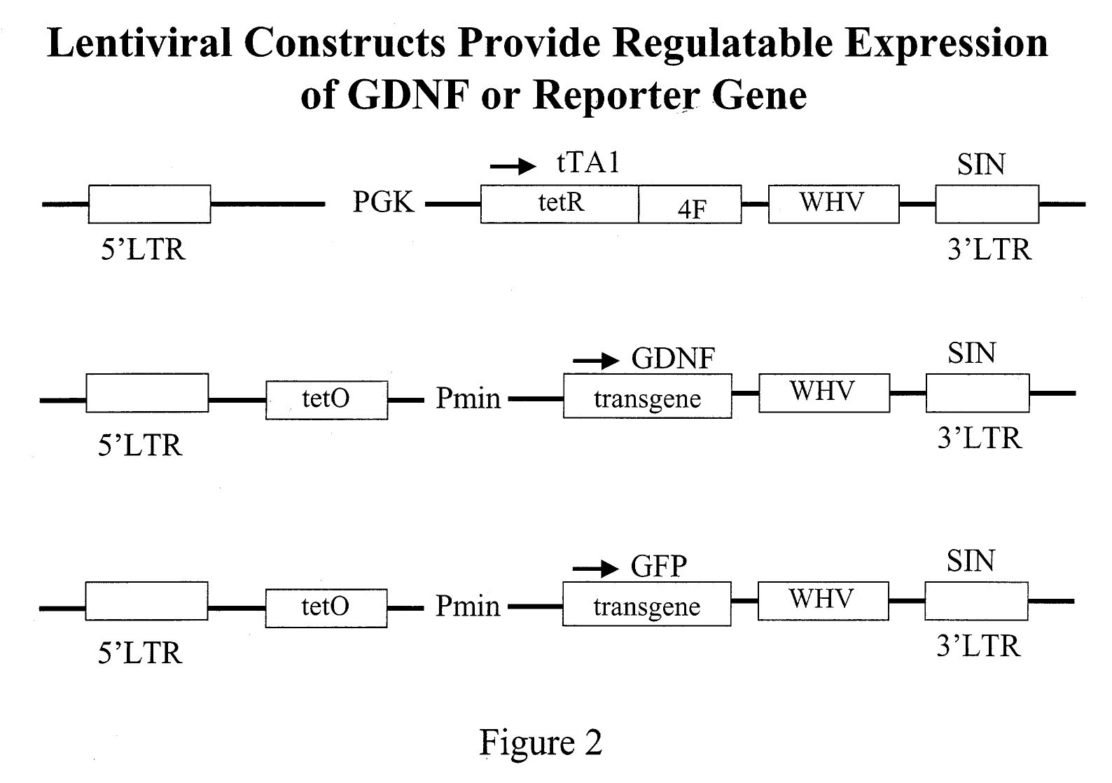

Preferably, our inducible lentiviral construct is based on the already published non-inducible system described in detail previously (Deglon, et al., Hum. Gene Ther. 11:179-190, 2000, incorporated by reference) and is shown schematically in FIG. 2. In this system, the mouse phosphoglycerate kinase 1 (PGK) promoter (strong constitutive promoter) drives the tTA1 in the lenti-tTA construct. The post-translational cis-acting regulatory element of the woodchuck hepatitis virus (WHV) is included and has been shown to significantly enhance transgene expression (Deglon, et al., supra, 2000). In the absence of doxycycline, tTA1 will bind to the tetO that is upstream of a minimal promoter driving the gene of interest (in this case GDNF in the ^{ind}lenti-GDNF construct or GFP in the of ^{ind}lenti-GFP construct). In the presence of DOX the tTA will be bound and not activate the transgene.

One of skill in the art could readily produce the GDNF gene sequence with reference to Genbank Accession Number L19063 and L15306 or Lin, et al., Science 260(5111):1130-1132, 1993, both incorporated by reference herein.

Cell growth and lentiviral infection. Human neural progenitor cells are maintained as neurospheres in DMEM/Ham's F12 supplemented with penicillin/streptomycin (1%), N2 (1%), and EGF (20 ng/ml). Neurospheres are chopped every 10 days, as diagramed in FIG. 1 and previously described (Svendsen, et al., J. Neuro. Meth. 85:141-153, 1998). The lentiviral particles were suspended in 1% fetal bovine serum albumin in phosphate buffered saline. Lentivirus infection was 6 hours with 25 ng of ^{ind}lenti-GFP or ^{ind}lenti-GDNF and 75 ng of lenti-tTA per sphere.

Immunocytochemistry. Neurospheres infected with ^{ind}lenti-GFP or ^{ind}lenti-GDNF were dissociated using ACCUTASE and plated onto glass coverslips coated with poly-L-lysine (0.01%) and laminin (0.001%). Cells were plated at 30,000 per coverslip in B27 differentiation media for 7 days. Following a 20 minute fixation with 4% paraformaldehyde and rinses with phosphate buffered saline, cells were stained for nestin (rabbit, Chemicon, 1:200), β-tubulin (mouse, Sigma, 1:6000), GFAP (rabbit, Dako, 1:3000) or GDNF (goat, R&D Systems, 1:2000) with fitc-conjugated secondary antibodies (Hoechst).

GFP regulation. Following ^{ind}lenti-GFP infection, the GFP expression in a representative neurosphere was demonstrated by a fluorescent photograph, and a phase photograph was taken at the same time. This sphere was then cultured in media with doxycycline (100 ng/ml) for 48 hours and again photographed under both fluorescence and phase. Doxycycline was removed from the media for 48 hours. Following this washout, a photograph was again taken under both fluorescence and phase.

GDNF quantification and regulation. Following ^{ind}lenti-GDNF infection, GDNF levels and regulation were assessed. Neurospheres (n=3) were individually dissociated with ACCUTASE and equally divided into 2 wells. One well was maintained in B27 differentiation medium and one well was maintained in B27 differentiation medium with doxycycline (1000 ng/ml). From the 6 wells (3 neurospheres divided into no DOX and yes DOX media), 1 ml of supernatant was collected every 2 days for 10 days. The plating medium with or without DOX was replenished every 48 hours, and the 1 ml samples were stored at −20° C. for later analysis. GDNF was measured in the sampled media and in media of ^{ind}lenti-GFP-infected neurospheres using a GDNF ELISA Kit (Promega), according to manufacturer's instructions. For each collection day, we report GDNF levels in the plus DOX groups as a percentage of the GDNF levels in the minus DOX groups. For the collection at two days following dissociation and plating, we report the GDNF level for each individual sphere divided into plus and minus DOX.

GDNF functional effects. Primary ventral mesencephalon was dissected from E14 embryos of Sprague-Dawley rats and plated onto poly-l-lysine, laminin-coated coverslips. Cells were cultured for 7 days in either basal N2 (1%) medium (n=3), supernatant from wild-type neurospheres (n=3) or supernatant from neurospheres infected with ^{ind}lenti-GDNF (n=3). Following a 20 minute fixation with 4% paraformaldehyde and rinses with phosphate buffered saline, cell cultures were stained for tyrosine hydroxylase (mouse, Chemicon, 1:200) with fitc-conjugated secondary antibodies (Hoechst). Cells were viewed under a fluorescent microscope and four fields were analyzed from each of the 3 coverslips per group. Fluorescent digital images were captured with a digital video camera using the SPOT camera image analysis system. The number of TH-positive cells was quantified by counting cells immunoreactive for TH in 12 randomly selected fields. The neurite length and cell body size was quantified by using metamorph to determine the μm and radius, respectively, for TH-positive cells in 12 randomly selected fields.

Results

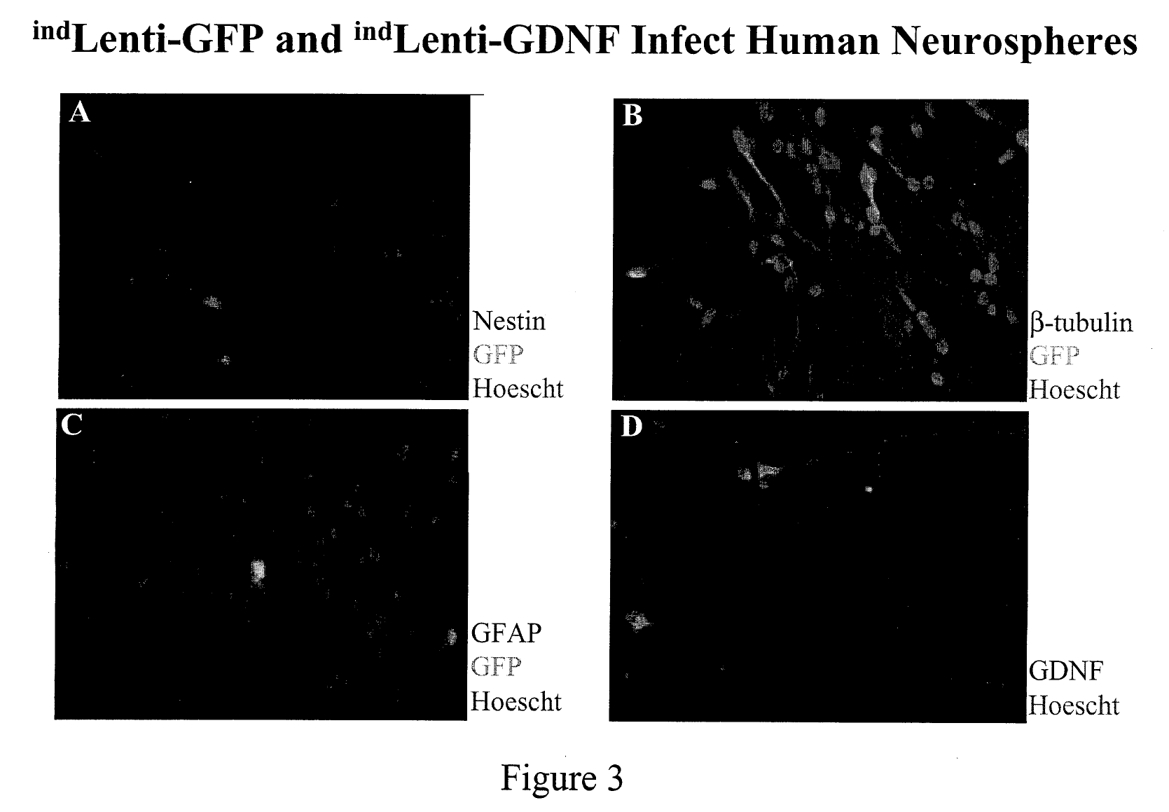

Lentiviral infection. Cells within the neurosphere were efficiently infected by the lentivirus constructs. The ^{ind}lenti-GFP construct was able to infect all cells types within the neurosphere, including progenitor cells, neurons and astrocytes (FIGS. 3A-C). The ^{ind}lenti-GDNF construct was also able to infect cells within the neurosphere (FIG. 3D). With both lentiviral constructs, infection did not affect cell health, shown by the normal cellular morphology of infected cells compared to the non-infected cells. Cells within the neurosphere continued to express GFP and GDNF for at least several months following infection.

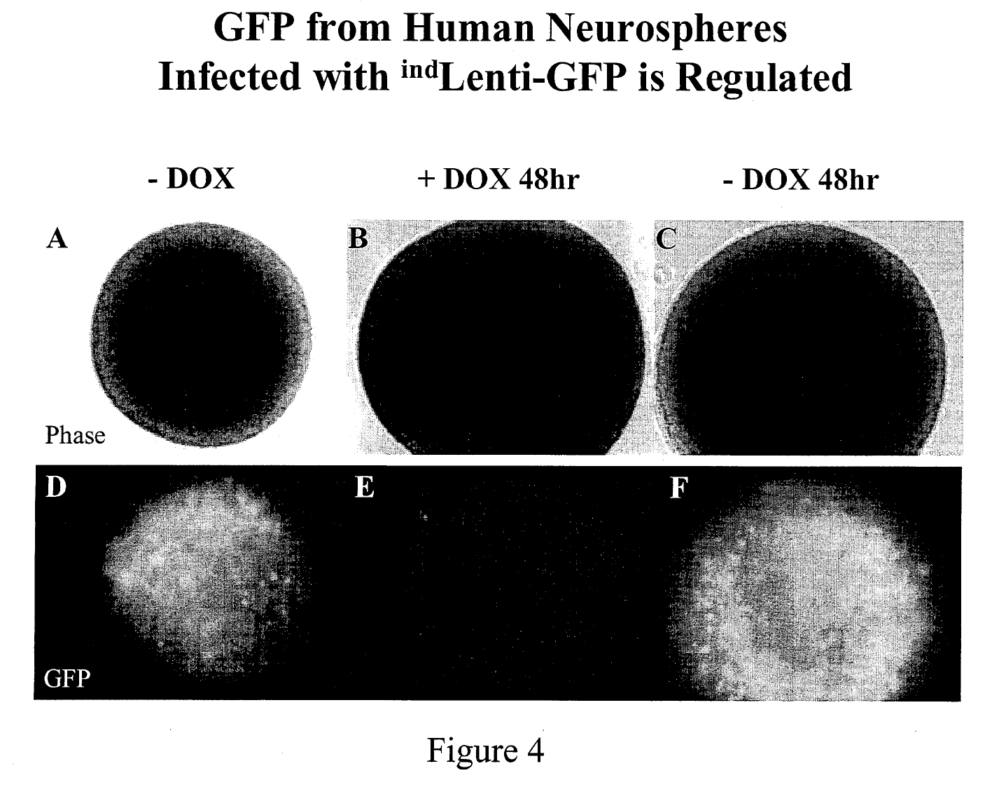

GFP regulation. GFP, unlike GDNF, is a protein that can be visualized in living cells. Therefore, we first used the ^{ind}lenti-GFP construct to optimize our methods of lentiviral infection of human cells and of regulation of gene expression. Co-infection of neurospheres with the ^{ind}lenti-GFP and lenti-tTA constructs resulted in a high percentage of GFP-expressing cells (FIG. 4D). When GFP-expressing neurospheres were grown in the presence of doxycycline for 48 hours, GFP was almost entirely shut-off (FIG. 4E). To further characterize this tight regulation of GFP, doxycycline was removed for 48 hours. After this brief washout, a robust expression of GFP resumed (FIG. 4F). Complementing the normal cell morphology following lentiviral infection, phase pictures of the GFP-expressing neurosphere show infection did not affect cell health, demonstrated by the continued normal growth of the neurosphere over time and by the typical healthy appearance (FIGS. 4A-C).

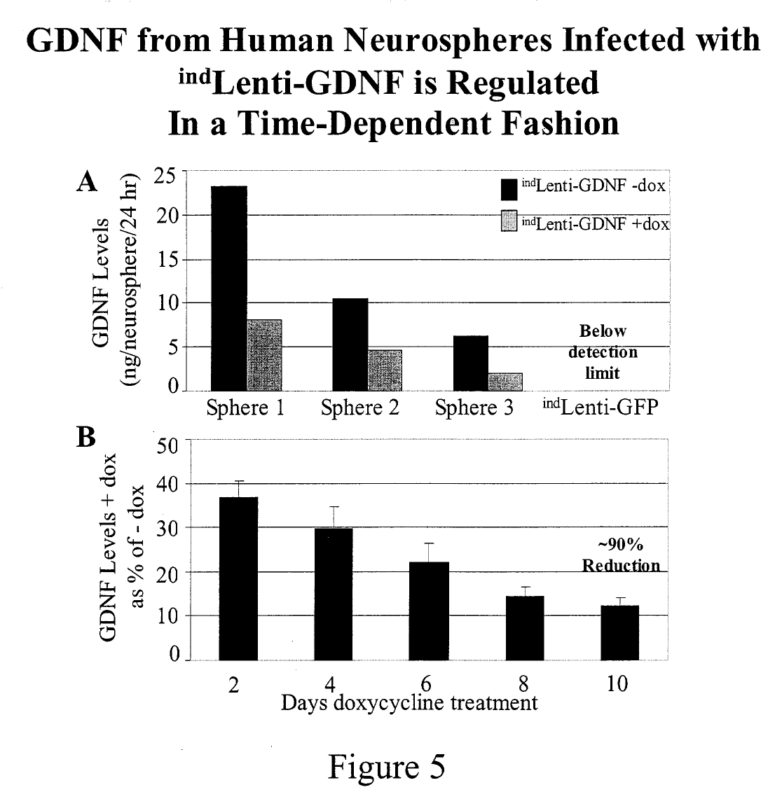

GDNF quantification and regulation. Having optimized lentiviral infection and regulation of human neural cells using the visible GFP reporter, we next co-infected neurospheres with the ^{ind}lenti-GDNF and lenti-tTA constructs. We found that neurospheres with ^{ind}lenti-GDNF released GDNF into the medium at high concentrations, ranging from 6 ng to 23 ng in 24 hours for one neurosphere (FIG. 5A). Neurospheres infected with lenti-GFP did not release GDNF at levels high enough for measurement even with sensitive detection methods (FIG. 5A). The range of GDNF levels released from individual neurospheres suggests the potential of selecting and propagating individual neurospheres with the highest gene expression. Interestingly, the degree of GDNF regulation was similar amongst the neurospheres regardless of differing GDNF levels. Following 2 days of DOX treatment, the range of decrease in GDNF levels was 56% to 68% compared to cells without DOX, with an average decrease of 64%. GDNF levels were reduced after 2 days of doxycycline treatment, and continued to decrease in a time-dependent fashion due to the long half-life of the GDNF protein. By 10 days of DOX treatment, there was an almost 90% decrease in GDNF levels compared to cells without DOX (FIG. 5B).

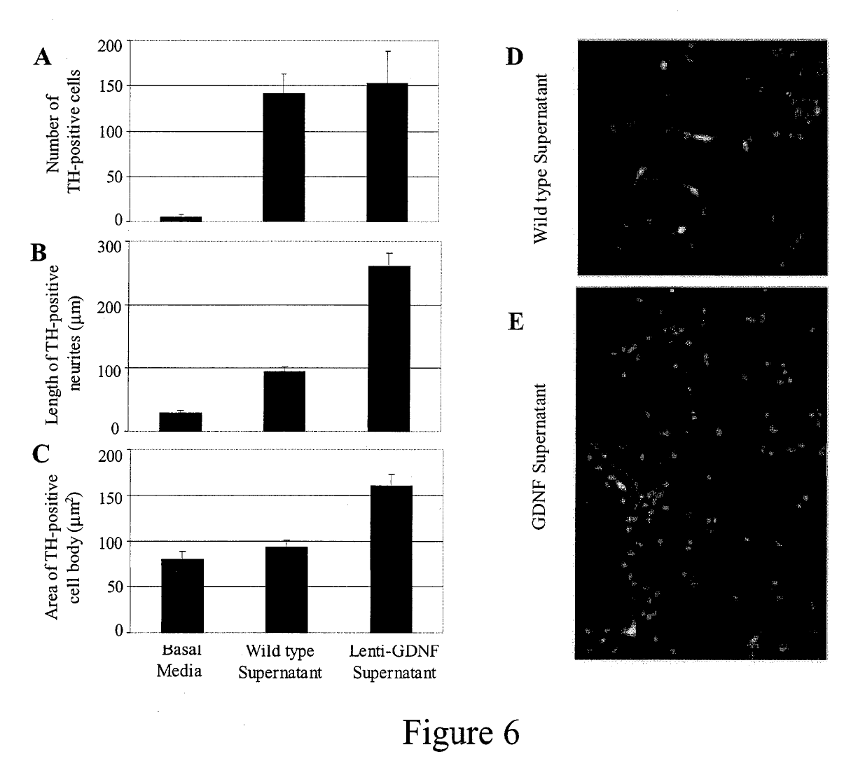

GDNF has a functional effect. Having shown that neurospheres infected with ^{ind}lenti-GDNF release high levels of GDNF, we next established the functional effects of these neurospheres on dopamine neurons. Primary dopamine neurons were cultured in either basal media, supernatant from wild-type human neurospheres or supernatant from ^{ind}lenti-GDNF infected neurospheres. Tyrosine hydroxylase (TH) is used as a marker for dopaminergic neurons. The number of TH-positive cells significantly increased when cultures were grown in supernatant from wild-type human neurospheres or supernatant from ^{ind}lenti-GDNF infected neurospheres compared to cultures grown in basal media (p<0.0001) (FIG. 6A). This suggests an overall effect of conditioned media on cell number that is not further increased by GDNF. Neurospheres infected with ^{ind}lenti-GDNF do, however, significantly affect the neurite length of cultured dopamine neurons (FIG. 6B). Dopamine neuron cultures grown in ^{ind}lenti-GDNF supernatant had significantly increased neurite outgrowth compared to both basal media and wild-type supernatant, demonstrating a functional effect of neurospheres infected with ^{ind}lenti-GDNF (p<0.0001). The neurite length of cultures grown in wild-type supernatant is far below that of cultures grown in ^{ind}lenti-GDNF supernatant, however, length is increased compared to cultures in basal media (p<0.0001), again suggesting some effect of conditioned media. In addition, neurospheres infected with ^{ind}lenti-GDNF significantly affect the cell body size of cultured dopamine neurons (FIG. 6C). Dopamine neuron cultures grown in ^{ind}lenti-GDNF supernatant had significantly increased cell body size compared to both basal media and wild-type supernatant, again demonstrating a functional effect of neurospheres infected with ^{ind}lenti-GDNF (p<0.0001). The cell body size of neurons grown in wild-type supernatant is not increased compared to cultures in basal media, suggesting no effect of conditioned media. The functional effects of ^{ind}lenti-GDNF infected neurospheres compared to wild-type neurospheres is clearly demonstrated by the increased neurite length and cell body size of dopamine neurons in FIGS. 6D and E. The fact that GDNF released from ^{ind}lenti-GDNF infected neurospheres has potent effects on cultured dopamine neurons demonstrates that these cells are releasing GDNF at physiologically relevant levels in vitro.

I claim:

1. A method of treating brain disorders involving loss of cells that respond to GDNF comprising the steps of
(a) transducing human neural stem cells with glial-derived neurotrophic factor (GDNF), wherein the GDNF gene is under control of an inducible promoter system, and
(b) transplanting the transduced cells into the brain of a patient, wherein GDNF is expressed.

2. The method of claim 1 wherein the patient's GDNF-responsive neuron system is up-regulated.

3. The method of claim 1 wherein the disorder is Parkinson's Disease.

4. The method of claim 3 wherein the GDNF-responsive neuron system is the dopaminergic system.

5. The method of claim 1 wherein the disorder is selected from the group consisting of Parkinson's Disease, amyotrophic lateral sclerosis, Huntington's Disease, and stroke.

6. The method of claim 1 wherein the inducible promoter system is the mouse phosphoglycerate kinase 1/tTA1 system.

7. The method of claim 1 wherein the human neural cells are grown as neurospheres.

8. The method of claim 1 wherein the cells are derived from post-mortem fetal brain tissue.

9. The method of claim 1 wherein the transplanted cells migrate and integrate into the patient's brain.

10. The method of claim 1 wherein the cells are transplanted into the brain putamen.

11. The method of claim 10 wherein the cells are transplanted into the caudal half of the brain putamen.

12. The method of claim 1 wherein the transduced cells comprise an additional heterologous growth factor.

13. A viral vector useful for the method of claim 1, wherein the viral vector comprises an inducible promoter and a sequence encoding GDNF.

14. The vector fo claim 13, wherein the vector is a lentivirus.

15. The vector of claim 14 wherein the vector comprises the mouse phosphoglycerate kinase 1 promoter operably connected to tTA1 and the post-translational cis-acting regulatory element of the woodchuck hepatitis virus.

16. The vector of claim 15 wherein administration of doxycycline would activate GDNF expression.

17. The vector of claim 15 wherein administration of doxycycline would inactivate GDNF expression.
